(19) United States
(12) Patent Application Publication
(10) Pub. No.: US 2016/0346300 A1
(43) Pub. Date: Dec. 1, 2016

(54) LIQUID FORMULATIONS OF CELECOXIB FOR ORAL ADMINISTRATION

(71) Applicant: KIEL LABORATORIES, INC., Flowery Branch, GA (US)

(72) Inventors: JEFFREY SCOTT KIEL, GAINESVILLE, GA (US); THOMAS JEFFREY BRYANT, WINDER, GA (US); RICHARD GERARD LeVASSEUR, OAKWOOD, GA (US); HUGH GREG THOMAS, CARROLLTON, GA (US)

(21) Appl. No.: 15/163,258

(22) Filed: May 24, 2016

Related U.S. Application Data
(60) Provisional application No. 62/168,012, filed on May 29, 2015.

(51) Int. Cl. A61K 31/635; A61K 9/00; A61K 47/02; A61K 47/10; A61K 47/36; A61K 9/08; A61K 47/26

(52) U.S. Cl. CPC A61K 31/635; A61K 9/08; A61K 9/0053; A61K 47/26; A61K 47/10; A61K 47/36; A61K 47/02

(57) ABSTRACT

The invention provides pharmaceutical preparations of celecoxib in suspension, solution, and combination thereof for oral administration. Insolubility of celecoxib in water is overcome in embodiments of the invention by employing co-solvents, and liquid dosage forms having celecoxib in solution at concentrations up to 10 mg/ml are provided. Manufacturing methods for celecoxib suspensions are included in the present disclosure.

### CROSS REFERENCE TO RELATED APPLICATION

This application claims priority to U.S. Provisional Application Ser. No. 62/168,012, filed May 29, 2015, which is herein incorporated by reference in its entirety.

### BACKGROUND

The present invention relates generally to aqueous formulations of celecoxib in solution and suspension form, and methods for manufacturing aqueous celecoxib formulations.

Celecoxib, a diaryl substituted pyrazole chemically designated as 4-(4-methylphenyl)-3-(trifluoromethyl)-1H-pyrazol-1-yl]benzenesulfonamide (FIG. 1), was first approved by the FDA as an oral capsule in 1998 under the tradename Celebrex® for the treatment of osteoarthritis and rheumatoid arthritis in adults. Celebrex® is also approved for treatment of acute pain and for treatment of Juvenile Rheumatoid Arthritis (JRA) in patients 2 years and older. Two Orange Book patents, U.S. Pat. No. 5,760,068 and U.S. Pat No. 5,563,165 for the celecoxib drug substance and its pharmaceutical composition are associated with the original new drug application (NDA 020998) sponsored by G. D. Searle & Company. Both Orange Book patents expired on May 30, 2014.

Oral drug solutions are often preferred for children, and also for adults who have difficulty swallowing pills. Furthermore, the ability to customize the dosage of the drug is another advantage of solution preparations that could be used for oral and/or parenteral administration. However, celecoxib's poor water solubility and wettability has hampered its solution formulation. Experimental aqueous formulations of celecoxib in suspension form have been reported, however, celecoxib particles are known to aggregate, thereby requiring use of the suspensions shortly after formulation. For example, U.S. Pat. No. 6,579,895 (the '895 patent) describes ethanol/polysorbate 80 suspensions of particulate celecoxib which are administered within 5 minutes after preparation. Pharmacy compounded suspensions (so-called “extemporaneous formulations”) of celecoxib have been prepared in ORA-Blend®, a commercialized oral suspending vehicle (Reference 1).

Otherwise, examples of celecoxib in aqueous solution are described by Agrawal (Reference 2). In particular, Agrawal reports four aqueous polyethylene glycol celecoxib compositions for injection, wherein the polyethylene glycol in the formulations is included as a co-solvent to increase celecoxib solubility. Two of the formulations also contain either urea or piperazine as solubility enhancers. The two formulations (CPEG6W and CPEG4W) without piperazine or urea solubility enhancers contain 27% (w/w) water and 73% (w/w) polyethylene glycol (calculated based on 35 mL PEG with q.s. 50 mL water for injection). Interestingly, the inactive ingredients (“IIG”) in the Aggrawal celecoxib solutions are not currently approved for use by the FDA in the United States. The IIG Limit for PEG 400 and PEG 600 is 5% for injectable solutions and 60% PEG400 and 13% PEG600 for oral concentrates. Piperazine is not in use in FDA approved injectable or oral solutions, and urea is only in preparations for intramuscular injections.

Otherwise, the '895 patent describes a comparative aqueous celecoxib formulation administered in a hard gelatin capsule. The '895 patent capsule formulation is a mixture of water (2.7% w/w), PEG400 (27.1%w/w), and Tween® 80 (21.7% w/w). The '895 patent capsule also contains the polymeric excipients HPMC and PVP, suggesting the capsule formulation is semi-solid, or a viscous solution.

Hence, there remains a need for liquid formulations of celecoxib that are suitable for oral administration.

### SUMMARY OF THE INVENTION

The presently disclosed invention provides, in a general aspect, aqueous celecoxib formulations. In one aspect, the invention comprises celecoxib in solution, suspension, or combination thereof and wherein the incorporation of co-solvents allows for liquid dosage forms of celecoxib at least up to concentrations of 10 mg/mL.

In one embodiment, the invention is a pharmaceutical preparation for use in humans and/or animals including (a) 5-10 mg/mL celecoxib in solution, suspension, or combination thereof; (b) a co-solvent; and (c) at least 50% w/w of water. In a further embodiment, the co-solvents are selected from among ethanol, glycerin, polyethylene glycol 400, polysorbate 80, polyoxyl 40 hydrogenated castor oil, a poloxamer, propylene glycol, and combinations thereof. In some embodiments, the pharmaceutical preparation is for oral administration. In further embodiments, celecoxib is in solution.

In other embodiments, the pharmaceutical preparation includes (a) 10 mg/mL celecoxib in solution, suspension, or combination thereof; (b) a co-solvent selected from among ethanol, glycerin, polyethylene glycol 400, polysorbate 80, a poloxamer, propylene glycol, and combinations thereof; (c) at least one non-ionic surfactant; and (d) at least 10% w/w of water.

In another aspect, the present disclosure provides aqueous formulations of celecoxib in suspension form, and methods for manufacturing the formulations. According to one embodiment of the present disclosure, an aqueous pharmaceutical preparation is provided comprising 0.1-2.5% celecoxib (w/v), 5-30% propylene glycol (w/v), 2.5-30% glycerin (w/v), 0.1-2.5% xanthan gum (w/v), optionally 0.2-2.5% magnesium aluminum silicate (w/v), at least 50% water, and a pH that is about 3 to about 7, wherein the aqueous formulation is chemically and physically stable after at least 3 months storage at 40° C. Methods for making the formulations as described are provided in further embodiments.

### BRIEF DESCRIPTION OF THE DRAWINGS

FIG. 1 shows the structure of celecoxib.

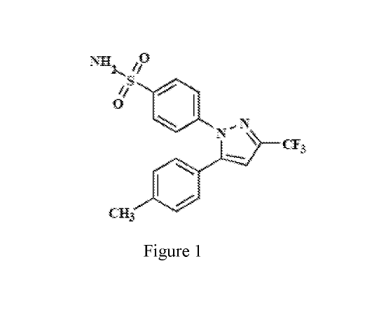

### DETAILED DESCRIPTION

The presently disclosed invention provides, in general, pharmaceutical preparations for use in humans and/or animals that are aqueous compositions of celecoxib that may be formulated for oral administration. In some embodiments, 5-10 mg of celecoxib is provided in a solution, suspension or combination thereof, and the formulation further includes one or more co-solvents, and at least 50% w/w of water. In some embodiments celecoxib is in solution. By soluble or “in solution”, it is meant that celecoxib is uniformly distributed throughout the formulation, is not visible to the naked eye as a solid, and does not settle out of the formulation as a solid upon standing. For solutions, analytical methods for quantifying the percent of dissolution may also be used, such as an HPLC method developed for determining the concentration of celecoxib from a sample that has been filtered to remove solids. Analytical determination and reporting of the amount of celecoxib in solution may be defined as a range such as ±10% of the formulated amount of celecoxib, so that being in solution is in such case defined as meaning that 90 to 110% of the formulated amount of celecoxib is determined to be dissolved in a formulation.

By “suspension” is meant a heterogeneous mixture containing solid (solute) particles, sometimes called the dispersed phase, in a dispersion medium such as a solvent or solvent mixture, wherein the solid particles will eventually settle out of a solution. In comparison, in solutions, the solute does not exist as a solid, and the mixture of the solute in the solvent is homogeneous. The settling time of a solute out of a liquid dispersion medium will depend, among other things, on the type and amount of suspending agent(s) in the composition. If not visible by the naked eye, particles in suspension are visible under a microscope. The sizes of the suspended particles according to the present invention are about 1 micron to about 200 microns. In some compositions the sizes of the particles are about 1 to about 100 microns.

Co-solvents are employed in the celecoxib preparations herein described to increase the solubility of celecoxib. Celecoxib is a hydrophobic molecule with low solubility in water. However, the solubility of celecoxib in aqueous preparations may be increased by using certain co-solvents. In some cases, synergistic combinations and amounts of co-solvents provide unexpected solubility and stability of celecoxib in an aqueous preparation than would be expected based on celecoxib solubility in the co-solvent alone, or based on its solubility in a single co-solvent plus water.

Preferred co-solvents are those that increase the solubility of celecoxib in aqueous compositions to the desired level of drug incorporation, are approved by the FDA for oral administration, and/or are generally regarded as safe (GRAS), and provide compositions that exhibit physicochemical stability. The stability of formulated compositions may be assessed by a stability program. Stability programs may be devised depending on the desired shelf life and the requirements for regulatory approval. A stability program may include evaluation of the stability of a composition at a variety of temperatures, wherein elevated temperature stability may be part of an accelerated stability testing program wherein stability at higher temperatures for a given time period is predictive of longer term stability at lower temperatures. Parameters that may be evaluated in a stability program include, for example, appearance, stability of the formulated ingredients, level of impurities which may arise due to degradation, physical properties such as specific gravity and viscosity, pH, and bioburden (i.e. levels of microorganisms).

In some embodiments, the presently disclosed invention provides liquid formulations for oral administration comprising 5 to 10 mg/mL celecoxib, a co-solvent, and at least 50% water (w/w). In a further embodiment, the co-solvents are selected from among ethanol, glycerin, polyethylene glycol 400, polysorbate 80, polyoxyl 40 hydrogenated castor oil (CO40), a poloxamer, propylene glycol, and combinations thereof. In further embodiments, celecoxib is in solution.

In other embodiments, the amount of celecoxib is 5 mg/mL and the co-solvents are a combination of PEG 400 and CO40. In one exemplary embodiment, the amount of PEG400 is about 21% (w/w), and the amount of CO40 is 10% (w/w).

In certain embodiments of the present disclosure, liquid formulations for oral administration are provided having (a) 10 mg/mL celecoxib in solution, suspension, or combination thereof; (b) a co-solvent that is selected from ethanol, glycerin, polyethylene glycol 400, a poloxamer, propylene glycol, and combinations thereof;(c) at least one non-ionic surfactant; and (d) at least 10% w/w of water. In some embodiments, the non-ionic surfactant is C40, polysorbate 80, or combinations thereof. In further embodiments, the nonionic surfactant is polysorbate 80 at a concentration of ≦10%. In some embodiments, the co-solvent includes polyethylene glycol at a concentration that is ≦62% (w/w). In other embodiments the co-solvent includes polyethylene glycol at a concentration that is ≦62% and a nonionic surfactant including polysorbate 80.

The present invention also includes aqueous pharmaceutical preparations of celecoxib for use in humans and/or animals, wherein celecoxib is suspended in the preparation. In some cases, celecoxib may be partially suspended. By partially suspended it is meant that at a portion of celecoxib in the preparation is suspended as a solid particle. A portion of celecoxib in the formulation that is not suspended may be dissolved in the formulation as a solution. One way of determining the portion of celecoxib that is suspended would be to analyze the amount of celecoxib in the formulation as compared to the amount of celecoxib in the formulation after it has been filtered to remove suspended celecoxib particles.

By means of certain combinations of particular types and amounts of ingredients, the celecoxib preparations described herein exhibit chemical and physical stability. In some embodiments, the preparations exhibit chemical and physical stability after at least 3 months storage at 40° C.

In an exemplary embodiment, the presently disclosed invention includes a pharmaceutical preparation for use in humans and/or animals containing (a) 0.1-2.5% suspended celecoxib (w/v); (b) 5-30% propylene glycol (w/v); 2.5-30% glycerin (w/v); 0.1-2.5% xanthan gum (w/v); at least 50% water, and a pH adjusted to between about 3 to about 7, and the preparation exhibits physical and chemical stability after at least 3 months storage at 40° C. In a further embodiment, the celecoxib suspension includes 0.1-2.0% citric acid (w/v) and 0.01-2.0% trisodium citrate, dihydrate (w/v). In another embodiment, the suspension includes sodium phosphate, monobasic, monohydrate, and sodium phosphate dibasic. In certain embodiments, the pH of the suspension is between about 4 to about 6. One exemplary embodiment includes 0.1-2.5% grape flavor. In one embodiment, the particle size of celecoxib is between about 1 micron to about 200 microns. In a further embodiment, 0.2-2.5% magnesium aluminum silicate (w/v) may be added. The instant inventors have discovered that it is sometimes desirable to not include magnesium aluminum silicate in large batches of the celecoxib preparation so as to facilitate pH adjustment and processing. In such cases, omission of magnesium aluminum silicate did not have any adverse effect on the final product properties.

In another exemplary embodiment of the invention, the amount of celecoxib is about 1% (w/v), the amount of propylene glycol is about 5% (w/v), the amount of glycerin is about 15%(w/v), the amount of xanthan gum is about 0.25, and the pH is 5.0 ±0.2. Magnesium aluminum silicate, such as in an amount that is about 1% (w/v) may be included in a further embodiment.

The presently disclosed invention also provides a method for preparing a celecoxib suspension. In a general aspect, the presently disclosed method comprises preparing a pre-mix composition wherein celecoxib is dispersed in a mixture of non-aqueous solvents and an emulsifying/suspending agent. The premix is added to an aqueous mixture of other formulation excipients including wetting agents/stabilizers, buffers, flavorings, and preservatives. The temperature of the pre-mix, amount of stirring time, and order of incorporation of the ingredients is selected so as to provide optimal dispersion and insure the stability of all of the formulation ingredients.

In one exemplary embodiment, a method for preparing a celecoxib suspension as has been generally described above comprises sequentially adding to a first vessel ingredients including propylene glycol, methylparaben, propylparaben, glycerin, optional magnesium aluminum silicate, xanthan gum, and celecoxib, and mixing after each ingredient is fully dissolved, in the case of propylene glycol, methylparaben, propylparaben, and glycerin, or for, xanthan gum and celecoxib and optionally, magnesium aluminum silicate, until they are fully dispersed. A portion of water is added to a second vessel, and the contents of the first vessel are added to the second. Buffers and/or flavorings are then added to the second vessel, and after determining the pH, the pH is adjusted with an acid or a base. The suspension batch is finished by adding quantum sufficit (q.s.) water to provide a final desired batch weight.

In some embodiments, the portion of water added to the second vessel is about 40% of the desired final batch weight. In another embodiment, the flavorings include sucralose. In another embodiment the flavoring further includes grape flavoring. Of course, other flavorings may be used according to the preference of the formulator and the consumer. In one embodiment, the buffer that is added to the suspension includes citric acid and sodium citrate.

To adjust the pH of the aqueous formulations described herein, one or both of an acid and/or base are used. In certain embodiments, the acid and base is citric acid and sodium citrate, respectively, which are desirable as a buffer acid/base pair for adjusting and maintaining pH between about 3.5 to about 5. In other embodiments, the acid and base may be other buffer components that provide the desired buffering strength and pH range for the formulation. For example, in some instances, phosphate buffer components such as sodium dihydrogen phosphate and disodium hydrogen phosphate may be used. Mixtures of different buffering types are another option, for example combinations of citric acid/citrate and sodium phosphates.

Preservatives may also be added to the aqueous formulations herein described. As will be understood by one skilled in the art, the function of a preservative or preservative mixture in a formulation is to provide a means for controlling and preventing escalation of microorganisms to unsafe levels during storage. Because preservative efficacy may be affected by the particular type and quantity of formulation components, preservative efficacy testing is a necessary regulatory requirement for packaged pharmaceutical products. In some embodiments of the presently disclosed invention, the preservatives include 0.015-0.2% methyl paraben and 0.01 to 0.1% propyl paraben.

In a further embodiment according to the present invention, a method for manufacturing a celecoxib suspension as previously described includes (a) preparing a first premix formulation combining ingredients consisting of propylene glycol, glycerin, methylparaben, propylparaben, xanthan gum, and celecoxib; optionally, (b) preparing a second premix formulation by combining water and magnesium aluminum silicate; (c) preparing an intermediate mixture by combining water, sucralose, citric acid, sodium citrate, and grape flavor together, and then adding the first and second premix formulations; and (d) preparing a final mixture by determining the pH of the intermediate mixture from step (c), adjusting the pH of the intermediate mixture using an acid or a base so that it is within a desired pH range, and adding q.s. amount of water. The method may further comprise heating the propylene glycol and glycerin mixture in step (a) to about 40 to about 45° C. prior to adding methylparaben and propylparaben, and discontinuing the heating after the methylparaben and propylparaben are fully dissolved.

In one exemplary embodiment, celecoxib is added to the first premix formulation after all of the other ingredients have been combined. As has been already generally described, in some embodiments, the acid or base used to adjust the pH of the formulation may be citric acid, or sodium citrate, respectively. Finally, the quantity of water in the preliminary formulations may be adjusted so as to provide dispersion and ensure stability of the components during manufacturing. In certain embodiments, the amount of water that is added to the second pre-mix in step (b) is about 40% of the total amount of water required for the formulation. In some embodiments, the amount of water that is added to the intermediate mixture in step (c) is about 20 to about 25% of the total amount of water required for the formulation.

### EXAMPLES

Below, the presently disclosed invention will be described by way of examples, which are provided for illustrative purposes only and accordingly are not to be construed as limiting the scope of the invention.

### Example 1

### Celecoxib Solutions

1.1 Co-Solvents.

A series of co-solvents were used for preparing aqueous solutions of celecoxib. All of the co-solvents are approved by the FDA for inclusion in orally administered drug products. The co-solvents included ethanol, polyethylene glycol 400 (PEG 400), Tween 80 (polysorbate 80), Kolliphor RH 40 (CO40), and Poloxamer.

1.2 Formulation Method.

Aqueous solutions of celecoxib were prepared by dissolving celecoxib in the co-solvent or combinations of co-solvents, and stirred until the solution was clear. The desired quantity of water was then added to the co-solvent solution.

1.3 Aqueous Celecoxib Compositions

Aqueous compositions of celecoxib (5 mg/mL and 10 mg/mL) were prepared according to the method described in 1.2, and are summarized in Table 1:

TABLE 1
Aqueous Compositions of Celecoxib

| | Composition # | | | | | | | | | |
|---|---|---|---|---|---|---|---|---|---|---|
| | A | B | C | D | E | F | G | H | I | J |
| | Celecoxib (mg/mL) | | | | | | | | | |
| | 5 | 10 | 10 | 10 | 10 | 10 | 10 | 5 | 5 | 5 |
| PEG 400 (% w/w) | | | 50 | 60 | 62 | 40 | 20 | | 21 | 21 |
| Tween 80 (% w/w) | | | | | | 10 | 10 | | | |
| Poloxamer (% w/w) | | | | | 1 | | | | | |
| Propylene Glycol (% w/w) | | | | 25 | | | | | | |
| Kolliphor RH40 (% w/w) | | | | | | | | 20 | 10 | 10 |
| Ethanol (% w/w) | 50 | 75 | 25 | 12.5 | 26 | | | | | |
| Water (% w/w) | 50 | 25 | 25 | 12.5 | 10 | 49 | 69 | 79.5 | 61.475 | 62.470 |

### Example 2

### Celecoxib Oral Solution (100 mg/20 mL)

An oral solution of 5 mg/mL celecoxib as shown and described in Table 2 was prepared. The solution specifications are described in Table 3

TABLE 2
Celecoxib Oral Solution (100 mg/20 mL)

| | | Quantity | | Weight | | | IIG |
|---|---|---|---|---|---|---|---|
| | | per | | per | | | max |
| | Quality | 100 L | | one | | | potency |
| INGREDIENT | Standard | Batch | | liter | | % w/v | %¹ |
| Celecoxib | USP | 0.5 | kg | 5 | g | 0.5% | NA |
| PEG 400 | USP | 21.0 | kg | 210 | g | 21.0% | 60% |
| Polyoxyl 40 | USP | 10 | kg | 100 | g | 10.0% | 45% |
| Hydrogenated | | | | | | | |
| Castor Oil | | | | | | | |
| (Kolliphor 40) | | | | | | | |
| Flavors | NA² | 4.7 | kg | 47.0 | g | 4.7% | NA |
| Sodium | NF | 0.2 | kg | 2 | g | 0.2% | 0.2% |
| methylparaben | | | | | | | |
| 0.8% | | | | | | | |
| Sucralose | NF | 0.1 | kg | 1 | g | 0.10% | 0.8% |
| Sodium | USP | 1 | kg | 10 | g | 1.0% | 1.1% |
| Citrate | | | | | | | |

| | | | | | |
|---|---|---|---|---|---|
| Purified Water | USP | QS | QS | QS | NA |

¹Inactive Ingredient;
²no monograph

TABLE 3
Preliminary Celecoxib Oral Solution Specifications

| Test/Method | Specifications |
|---|---|
| Appearance and | Clear liquid, with characteristic odor of grape |
| Organoleptic properties | |
| Identification | The retention time of the major peak of the |
| (STP-025) | Sample solution corresponds to that of the |
| | Standard solutions, as obtained in the Assay. |
| Assay (STP-025) | Celecoxib: 90.0%-110.0% |
| | Sodium Methylparaben: 85.0%-115.0% |
| Organic Impurities | Individual: NMT 0.2% |
| (STP-025) | |
| pH USP <791> | 5.5-6.5 |
| Specific Gravity | 0.950-1.050 |
| (USP <841>) | |
| Microbial | Total aerobic: NMT 200 cfu/mL; Molds & |
| Enumeration Tests | Yeast: NMT 20 cfu/mL; Escherichia coli: |
| USP <61>, | Absent. |
| <62>, <1111> | |

### Example 3

### Celecoxib Oral Suspensions at pH 3, 5, and 7

500 g batches of celecoxib in suspension, at three different pH's (3, 5, and 7) as shown in Tables 4A-4C were prepared. The suspensions were prepared according to Suspension Preparation Method I (Example 5.1).

TABLE 4A
Celecoxib suspension, 500 mL batch, pH 3.

| | | | Amount | |
|---|---|---|---|---|
| | | | per 500 | Actual |
| | IIG | % | mL Batch | Amount |
| Ingredient | Limit | (w/v) | (g) | (g) |
| Celecoxib, USP | n/a | 1.00% | 5.000 | 5.0005 |
| Magnesium Aluminum | 2% | 1.00% | 5.000 | 5.0120 |
| Silicate, NF | | | | |
| Xantham Gum, NF | 1.38% | 0.25% | 1.250 | 1.2564 |
| Citric Acid, anhydrous, | 0.71% | 0.20% | 1.000 | 1.0205 |
| USP | | | | |
| Trisodium Citrate, | 0.38% | 0.05% | 0.250 | 0.2517 |
| dihydrate, USP | | | | |
| Sodium Phosphate, | 0.75% | N/A | 0.000 | N/A |
| Monobasic, monohydrate | | | | |
| Sodium Phosphate, Dibasic | 0.95- | N/A | 0.000 | N/A |
| | 1.25% | | | |
| Natural Grape Flavor | n/a | 0.30% | 1.5000 | 1.53 |
| Glycerin, USP | 36.48% | 15.00% | 75.000 | 75.06 |
| Propylene Glycol, USP | 28.50% | 5.00% | 25.000 | 25.15 |
| Sucralose, NF | 1.10% | 0.10% | 0.500 | 0.5035 |
| Methylparaben, NF | 20% | 0.15% | 0.750 | 0.7521 |
| Propylparaben, NF | 4% | 0.05% | 0.250 | 0.2502 |
| Purified Water, USP | n/a | q.s. | q.s. | q.s. |

TABLE 4B
Celecoxib suspension, 500 mL batch, pH 5.

| | | | Amount | |
|---|---|---|---|---|
| | | | per 500 | Actual |
| | IIG | % | mL Batch | Amount |
| Ingredient | Limit | (w/v) | (g) | (g) |
| Celecoxib, USP | n/a | 1.00% | 5.000 | 5.0269 |
| Magnesium Aluminum | 2% | 1.00% | 5.000 | 5.0010 |
| Silicate, NF | | | | |
| Xantham Gum, NF | 1.38% | 0.25% | 1.250 | 1.2505 |
| Citric Acid, anhydrous, | 0.71% | 0.20% | 1.000 | 1.0099 |
| USP | | | | |
| Trisodium Citrate, | 0.38% | 0.05% | 1.000 | 1.0097 |
| dihydrate, USP | | | | |
| Sodium Phosphate, | 0.75% | N/A | 0.000 | N/A |
| Monobasic, monohydrate | | | | |
| Sodium Phosphate, Dibasic | 0.95- | N/A | 0.000 | N/A |
| | 1.25% | | | |
| Natural Grape Flavor | n/a | 0.30% | 1.500 | 1.70 |
| Glycerin, USP | 36.48% | 15.00% | 75.000 | 75.02 |
| Propylene Glycol, USP | 28.50% | 5.00% | 25.000 | 25.50 |
| Sucralose, NF | 1.10% | 0.10% | 0.500 | 0.5062 |
| Methylparaben, NF | 20% | 0.15% | 0.750 | 0.7512 |
| Propylparaben, NF | 4% | 0.05% | 0.250 | 0.2502 |
| Purified Water, USP | n/a | q.s. | q.s. | q.s. |

TABLE 4C
Celecoxib suspension, 500 mL batch, pH 7.

| | | | Amount | |
|---|---|---|---|---|
| | | | per 500 | Actual |
| | IIG | % | mL Batch | Amount |
| Ingredient | Limit | (w/v) | (g) | (g) |
| Celecoxib, USP | n/a | 1.00% | 5.000 | 5.0177 |
| Magnesium Aluminum | 2% | 1.00% | 5.000 | 5.0076 |
| Silicate, NF | | | | |
| Xantham Gum, NF | 1.38% | 0.25% | 1.250 | 1.2503 |
| Citric Acid, anhydrous, | 0.71% | N/A | N/A | N/A |
| USP | | | | |
| Trisodium Citrate, | 0.38% | N/A | N/A | N/A |
| dihydrate, USP | | | | |
| Sodium Phosphate, | 0.75% | 0.20 | 1.000 | 1.0090 |
| Monobasic, monohydrate | | | | |
| Sodium Phosphate, Dibasic | 0.95- | 0.15 | 0.750 | 0.7552 |
| | 1.25% | | | |
| Natural Grape Flavor | n/a | 0.30% | 1.500 | 1.50 |
| Glycerin, USP | 36.48% | 15.00% | 75.000 | 75.01 |
| Propylene Glycol, USP | 28.50% | 5.00% | 25.000 | 25.11 |
| Sucralose, NF | 1.10% | 0.10% | 0.500 | 0.5035 |
| Methylparaben, NF | 20% | 0.15% | 0.75 | 0.7513 |
| Propylparaben, NF | 4% | 0.05% | 0.25 | 0.2501 |
| Purified Water, USP | n/a | q.s. | q.s. | q.s. |

Celecoxib Suspension Stability Testing

The stability of celecoxib oral suspensions was evaluated using an HPLC method as described in Table 5. The dissolution of celecoxib oral suspension, pH 5, is summarized in Table 6.

TABLE 5
HPLC Method for Stability Testing

| Mobile Phase | 75% Methanol/25% Water | |
|---|---|---|
| Column Temp | 60° | C. |
| Column | Luna 5 μm Phenyl-Hexyl 250 mm × 4.6 mm | |
| | (USP L11) | |
| Diluent | 75% Methanol/25% Water | |
| Flow Rate | 1.5 | mL/min |
| Injection Volume | 25 | μL |
| Std/sample conc. | 0.05 | mg/mL |
| Wavelength | 250 | nm |

TABLE 6
HPLC Method Dissolution Time Analysis of Celecoxib Suspension

| | % Celecoxib by HPLC according | | |
|---|---|---|---|
| | to sample dissolution time^{1,2} | | |
| Formulation pH | 20 min | 40 min | 60 min |
| pH 5 | 98.2 | 99.3 | 98.8 |

¹Dissolution in 0.04M Sodium Phosphate, Monobasic, pH 12; Paddles at 75 rpm
²HPLC conditions as shown in Table 6, except diluent is 0.04M Sodium Phosphate, Monobasic

The stability of the Celecoxib Oral Suspension from Tables 4A-4C is summarized below in Table 7.

TABLE 7
HPLC Stability Test Results for Celecoxib Suspensions.

| | | 40° C. | 40° C. | 40° C. |
|---|---|---|---|---|
| pH | Initial¹ | 1 month¹ | 2 months¹ | 3 months² |
| 3 | 99.3 | 94.6 | 101.2 | 95 |
| 5 | 101.5 | 95.9 | 106.0 | 93.9 |
| 7 | 99.1 | 103.3 | 104.7 | 91.8 |

¹Sample concentration 0.05 mg/mL;
²Sample concentration 0.5 mg/mL.

### Example 4

### Celecoxib Oral Suspension

4.1 Suspension Formulation

An oral suspension of celecoxib (10 mg/mL) as described in Table 8 was prepared according to the processes as described in Example 5.

TABLE 8
Celecoxib Oral Suspension, 10 mg/mL

| | IIG | % | Amount | | Amount per | |
|---|---|---|---|---|---|---|
| Ingredient | Limit | (w/v) | per 1 mL | | 100 L Batch | |
| Celecoxib, USP | n/a | 1.0% | 10 | mg | 1.0 | KG |
| Magnesium Aluminum | 2% | 1.0% | 10 | mg | 1.0 | Kg |
| Silicate, NF | | | | | | |
| Xanthan Gum, NF | 1.375% | 0.25% | 2.5 | mg | 0.25 | Kg |
| Citirc Acid, | 0.7119% | 0.2% | 2 | mg | 0.2 | Kg |
| anhydrous, USP | | | | | | |
| Trisodium Citrate, | 0.3807% | 0.05% | 0.5 | mg | 0.05 | Kg |
| dehydrate, USP | | | | | | |
| Natural Grape Flavor | n/a | 0.3% | 3 | mg | 0.3 | Kg |
| (FONA code # | | | | | | |
| 856.0172U)36.48 | | | | | | |
| Glycerin, USP | 36.48% | 15.0% | 150 | mg | 15.0 | Kg |
| Propylene Glycol, USP | 28.5% | 5.0% | 50 | mg | 5.0 | Kg |
| Sucralose, NF | 1.1% | 0.1% | 1 | mg | 0.1 | Kg |
| Methylparaben, NF | 20% | 0.15% | 1.5 | mg | 0.15 | Kg |
| Propylparaben, NF | 4% | 0.05% | 0.5 | mg | 0.05 | Kg |

| | | | | |
|---|---|---|---|---|
| Purified Water, USP | n/a | q.s. | q.s. | q.s. |
| | | | (approx. | (approx. |
| | | | 770 mg) | 77.0 Kg) |

### Example 5

### Processes for Preparation of Celecoxib Oral Suspension

5.1 Suspension Preparation Method I

Step 1
Add propylene glycol “PG” (15 Kg) and glycerin (5 Kg) to a 10-15 gallon tank
Heat to 40-45° C.
Add methylparaben (0.15 Kg)
Add propylparaben (0.05 Kg)
Mix until fully dissolved
Turn the heat on the hot plate off
Add xanthan gum (0.25 Kb)
Mix until fully dispersed
Add celecoxib (1 Kg)
Mix until fully dispersed

Step 2

(may be performed concurrently with Step 1 above)
Add approximately 40 Kg of water to the main 100 L tank
Add sucralose, citric acid, sodium citrate, and grape flavor (0.65 Kg total)
Mix until fully dissolved

Step 3
Transfer the PG/Glycerin/Methylparaben/Propylparaben/Xanthan Gum/Celecoxib mixture from the 10-15 gallon tank and transfer rinsate to ensure entire contents have been transferred
Begin mixing the contents of the main tank

Step 4
Add approximately 20-25 Kg water to the 10-15 gallon tank
Heat to approximately 75° C.
Add magnesium aluminum silicate (1 Kg)
Mix for 45 minutes at 75° C.

Step 5
Transfer the magnesium aluminum silicate slurry to the 100 L main mixing tank
Mix until uniform

Step 6
Determine the pH; adjust if necessary with citric acid or sodium citate
Q.S. the mixture to 100 L
Mix until uniform
Confirm final pH

5.2 Suspension Preparation Method II

Step 1
Add propylene glycol “PG” (15 Kg) to a 10-15 gallon tank (Vessel 1)
Add methylparaben (0.15 Kg)
Mix until fully dissolved
Add propylparaben (0.05 Kg)
Mix until fully dissolved
Add glycerin (5 Kg)
Mix until homogeneous
Add magnesium aluminum silicate (1 Kg)
Mix until fully dispersed
Add xanthan gum (0.25 Kb
Mix until fully dispersed
Add celecoxib (1 Kg)
Mix until well dispersed

Step 2

(may be performed concurrently with Step 1 above)
Add approximately 40 Kg of water to the main 100 L tank (Vessel 2)
While mixing, quantitatively transfer the contents of Vessel 1 into Vessel 2.
Begin vigorous mixing.
Add sucralose, citric acid, sodium citrate, and grape flavor (0.65 Kg total)
Mix until fully dissolved

Step 3
Determine the pH; adjust if necessary with citric acid or sodium citrate
Q.S. the mixture to 100 L
Mix until uniform
Confirm final pH

### Example 6

### Celecoxib Oral Suspension—200 Liter Batch

A 200 liter batch of celecoxib oral suspension was prepared as described in Table 9

TABLE 9
Celecoxib Oral Suspension, Lot E0337

| | | |
|---|---|---|
| Raw Material | Quantity | % (w/v) |

| | | | |
|---|---|---|---|
| Purified Water - 1^{st} addition | 80.00 | kg | 40 |
| Citric Acid | 0.80 | kg | 0.4 |
| Sucralose | 0.20 | kg | 0.1 |
| Sodium citrate dihydrate | 2.2 | kg | 1.10 |
| Propylene glycol | 10.00 | kg | 5.00 |
| Methyl paraben | 0.30 | kg | 0.15 |
| Propyl paraben | 0.10 | kg | 0.05 |
| Glycerin | 30.00 | kg | 15.00 |
| Xanthan gum | 0.50 | kg | 0.25 |
| Celecoxib | 2.00 | kg | 1.00 |
| Grape Flavor | 0.6 | kg | 0.30 |
| Purified Water - q.s. addition | 200 | L | q.s. |

Procedure for Preparing 200 Liter batch of Celecoxib Oral Suspension (Lot E0337)
Step 1: 1. In small tank, add water and start agitation. 2. Add citric acid and mix until completely dissolved. 3. Add sucralose and mix until completely dissolved. 4. Add sodium citrate dihydrate and mix until completely dissolved.
Step 2: 1. Tare another tank (Faby tank) and transfer solution from step 1 into Faby tank.
Step 3: 1. In the small tank, add propylene glycol and start agitation. 2. Add methyl paraben and mix until completely dissolved. 3. Add propyl paraben and mix until completely dissolved.
Step 4: Add glycerin to the solution of step 3, and mix for at least 10 minutes.
Step 5: Add xanthan gum to the solution from step 4, while maintaining vigorous agitation until complete dispersion.
Step 6: To the solution from step 5, add celecoxib and mix until complete dispersion.
Step 7: In the Faby tank, start agitation and slowly transfer the solution from step 6. Mix with re-circulation for a minimum of 15 minutes. Rinse the tank and transfer pail with ˜2 liters purified water.
Step 8: To the solution from step 7, add grape flavor and mix at least 15 minutes until complete dispersion.
Step 9: Measure pH and adjust to 4.9-5.1 pH with 10% w/w NaOH or 10% HC1 solution for 5 minutes after each addition.
Step 10: QS with purified water to 200 L and mix for at least 60 minutes.
Step 11: Collect a sample and measure the pH (target =5.0±0.2).

### REFERENCES

1. Agrawal et al., IJPSR, 2012; Vol. 3(7):2325-2336.
2. Donnelly et al., Can J Hosp Pharm 2009;62(6):464-468.

What is claimed is:

1. A liquid pharmaceutical preparation for use in humans and/or animals comprising:
(a) 5-10 mg/mL celecoxib in solution, suspension, or combination thereof;
(b) at least one co-solvent; and
(c) at least 50% w/w of water.

2. The liquid pharmaceutical preparation according to claim 1 wherein celecoxib is in solution.

3. The liquid pharmaceutical preparation according to claim 1 wherein said use comprises oral administration.

4. The liquid pharmaceutical preparation according to claim 1, wherein said preparation exhibits chemical and physical stability after at least 3 months storage at 40° C.

5. The liquid pharmaceutical preparation according to claim 1, wherein said co-solvent is selected from ethanol, glycerin, polyethylene glycol 400, polysorbate 80, polyoxyl 40 hydrogenated castor oil, a poloxamer, propylene glycol, and combinations thereof.

6. The liquid pharmaceutical preparation according to claim 1 wherein the amount of celecoxib is 5 mg/mL, and the co-solvent consists of a combination of polyethylene glycol 400 and polyoxyl 40 hydrogenated castor oil.

7. The liquid pharmaceutical preparation according to claim 6, wherein the amount of polyethylene glycol 400 is about 21% (w/w), and the amount of polyoxyl 40 hydrogenated castor oil is about 10% (w/w).

8. The liquid pharmaceutical preparation according to claim 7, wherein celecoxib is in solution.

9. A liquid pharmaceutical preparation for use in humans or animals comprising:
(a) 10 mg/mL celecoxib in solution, suspension, or combination thereof;
(b) a co-solvent selected from among ethanol, glycerin, polyethylene glycol 400, a poloxamer, propylene glycol, and combinations thereof;
(c) at least one non-ionic surfactant; and
(d) at least 10% (w/w) of water.

10. A liquid pharmaceutical preparation according to claim 9 wherein said use comprises oral administration.

11. A liquid pharmaceutical preparation according to claim 9, wherein said preparation exhibits chemical and physical stability after at least 3 months storage at 40° C.

12. A liquid pharmaceutical preparation according to claim 9, wherein said co-solvent comprises polyethylene glycol at a concentration that is ≦62% (w/w).

13. A liquid pharmaceutical preparation according to claim 9, wherein said non-ionic surfactant is selected from among polyoxyl 40 hydrogenated castor oil, polysorbate 80, and combinations thereof.

14. A liquid pharmaceutical preparation according to claim 13, wherein said nonionic surfactant is polysorbate 80 at a concentration of ≦10 (w/w)%.

15. A liquid pharmaceutical preparation according to claim 9, wherein celecoxib is in solution, the co-solvent comprises ≦62% polyethylene glycol 400, and said nonionic surfactant comprises polysorbate 80.

16. A pharmaceutical preparation for use in humans and/or animals comprising:
(a) 0.1-2.5% (w/v) suspended celecoxib;
(b) 5-30% propylene glycol (w/v);
(c) 2.5-30% glycerin (w/v);
(d) 0.1-2.5% xanthan gum (w/v);
(e) at least 50% water; and
(f) a pH that is between about 3 to about 7; and
wherein said preparation exhibits chemical and physical stability after at least 3 months storage at 40° C.

17. The pharmaceutical preparation according to claim 16, further comprising 0.2-2.5% magnesium aluminum silicate.

18. The pharmaceutical preparation according to claim 16, wherein said use comprises oral administration.

19. The pharmaceutical preparation according to claim 16 further comprising 0. 1-2.0% citric acid (w/v) and 0.01% -2.0% (w/v) trisodium citrate, dihydrate.

20. The pharmaceutical preparation according to claim 16 further comprising sodium phosphate, monobasic, monohydrate, and sodium phosphate dibasic.

21. The pharmaceutical preparation according to claim 16 wherein the pH is about 4 to about 6.

22. The pharmaceutical preparation according to claim 16 further comprising 0.1-2.5% grape flavor.

23. The pharmaceutical preparation according to claim 16, wherein the celecoxib particle size is between about 1 micron to about 200 microns.

24. The pharmaceutical preparation according to claim 16 wherein the amount of celecoxib is about 1% (w/v), the amount of propylene glycol is about 5% (w/v), the amount of glycerin is about 15% (w/v), the amount of xanthan gum is about 0.25%, and the pH is 5.0±0.2.

25. The pharmaceutical preparation according to claim 24, further comprising 1% (w/v) magnesium aluminum silicate.

26. A method for manufacturing a celecoxib suspension comprising the steps of:
(a) sequentially adding to a first vessel the ingredients comprising (1) propylene glycol, (2) methylparaben, (3) propylparaben, (4) glycerin, optionally (5) magnesium aluminum silicate, (6) xanthan gum, and (7) celecoxib, and mixing after each ingredient is added until it is fully dissolved, in the case of ingredients 1-4, or fully dispersed in the case of ingredients 5-7;
(b) adding a portion of water to a second vessel, and quantitatively transferring the contents of the first vessel to the second vessel;
(c) adding one or more buffers and/or flavorings to the second vessel;
(d) determining the pH, and adjusting the pH with an acid or a base;
(e) adding q.s. water to provide a desired final suspension batch weight.

27. The method according to claim 26, wherein the amount of water added in step (b) is about 40% of the desired final suspension batch weight.

28. The method according to claim 26, wherein the acid and base comprise HC1 and NaOH, respectively.

29. The method according to claim 26, wherein said buffers comprise citric acid and sodium citrate.

30. The method according to claim 26, wherein said flavorings comprise sucralose.

31. The method according to claim 30, further comprising grape flavoring.
